(19) United States
(12) Patent Application Publication
(10) Pub. No.: US 2010/0198250 A1
(43) Pub. Date: Aug. 5, 2010

(54) ENDOVASCULAR PROSTHESIS DELIVERY SYSTEM

(76) Inventors: Donald R. Ricci, Vancouver (CA); Thomas R. Marotta, Etobicoke (CA); Gyasi Bourne, Vancouver (CA); Alexei Marko, Vancouver (CA); Ian McDougall, Vancouver (CA); Elliot Hong, Vancouver (CA)

Correspondence Address: KATTEN MUCHIN ROSENMAN LLP;(C/O PATENT ADMINISTRATOR), 2900 K STREET NW, SUITE 200, WASHINGTON, DC, 20007-5118, US

(21) Appl. No.: 12/754,597

(22) Filed: Apr. 5, 2010

Related U.S. Application Data
(63) Continuation of application No. 10/450,512, filed on Jun. 14, 2004, now abandoned.
(60) Provisional application No. 60/255,381, filed on Dec. 15, 2000.

(51) Int. Cl. A61M 29/00

(52) U.S. Cl. 606/192; 604/96.01

(57) ABSTRACT

An expandable dilation catheter advantageously useful to deliver and orient an endovascular prosthesis with respect to a target body passageway. The catheter comprises a first tubular member disposed in a proximal portion of the portion of the catheter and a second tubular member disposed in a distal portion of the catheter. The first tubular member and the second tubular member are in a spaced relationship with respect to one another. An expandable member (e.g., a balloon) is disposed distally of the second tubular member. A first lumen and a second lumen disposed in each of the first tubular member and in the second tubular member. The first lumen is in communication with an interior of the expandable member to function as an inflation lumen and the second lumen serves to receive a first guidewire. The first tubular member and second tubular member are interconnected by a coupling member.

This is a continuation application of U.S. patent application Ser. No. 10/450,512, filed Jun. 14, 2004, which is a 371 of PCT/CA01/01767, filed Dec. 14, 2001, which claims benefit of Ser. No. 60/255,381, filed Dec. 15, 2000, the contents of which are hereby incorporated herein.

### TECHNICAL FIELD

In one of its aspects, the present invention relates to an expandable dilation catheter. In another of its aspects, the present invention relates to a balloon dilation catheter. In yet another of its aspects, the present invention relates to a catheterization kit. In yet another of its aspects, the present invention relates to an endovascular prosthesis-mounted balloon dilation catheter.

### BACKGROUND ART

As is known in the art, an aneurysm is an abnormal bulging outward in the wall of an artery. In some cases, the bulging may be in the form of a smooth bulge outward in all directions from the artery—this is known as a “fusiform aneurysm”. In other cases, the bulging may be in the form of a sac arising from one side of the artery—this is known as a “saccular aneurysm”.

While aneurysms can occur in any artery of the body, it is only those which occur in the brain which lead to the occurrence of a stroke. Most saccular aneurysms which occur in the brain have a neck which extends from the cerebral blood vessel and broadens into a pouch which projects away from the vessel.

The problems caused by such aneurysms can occur in several different ways. For example, if the aneurysm ruptures, blood enters the brain or the subarachnoid space (i.e., the space closely surrounding the brain)—the latter is known as aneurysmal subarachnoid hemorrhage. This followed by one or more of the following symptoms: nausea, vomiting, double vision, neck stiffness and loss of consciousness. Aneurysmal subarachnoid hemorrhage is an emergency medical condition requiring immediate treatment. Indeed, 10-15% of patients with the condition die before reaching the hospital for treatment. More than 50% of patients with the condition will die within the first thirty days after the hemorrhage. Of those patients who survive, approximately half will suffer a permanent stroke. It is typical for such a stroke to occur one to two weeks after the hemorrhage itself from vasospasm in cerebral vessels induced by the subarachnoid hemorrhage. Aneurysms also can cause problems which are not related to bleeding although this is less common. For example, an aneurysm can form a blood clot within itself which can break away from the aneurysm and be carried downstream where it has the potential to obstruct an arterial branch causing a stroke. Further, the aneurysm can also press against nerves (this has the potential of resulting in paralysis or abnormal sensation of one eye or of the face) or the adjacent brain (this has the potential of resulting in seizures).

Given the potentially fatal consequences of the aneurysms, particularly brain aneurysms, the art has addressed treatment of aneurysms using various approaches.

Generally, aneurysms may be treated from outside the blood vessels using surgical techniques or from the inside using endovascular techniques (the latter falls under the broad heading of interventional (i.e., non-surgical) techniques).

Surgical techniques usually involve a craniotomy requiring creation of an opening in the skull of the patient through which the surgeon can insert instruments to operate directly on the brain. In one approach, the brain is retracted to expose the vessels from which the aneurysm arises and then the surgeon places a clip across the neck of the aneurysm thereby preventing arterial blood from entering the aneurysm. If there is a clot in the aneurysm, the clip also prevents the clot from entering the artery and obviates the occurrence of a stroke. Upon correct placement of the clip the aneurysm will be obliterated in a matter of minutes. Surgical techniques are the most common treatment for aneurysms. Unfortunately, surgical techniques for treating these conditions are regarded as major surgery involving high risk to the patient and necessitate that the patient have strength even to have a chance to survive the procedure.

As discussed above, endovascular techniques are non-surgical techniques and are typically performed in an angiography suite using a catheter delivery system. Specifically, known endovascular techniques involve using the catheter delivery system to pack the aneurysm with a material which prevents arterial blood from entering the aneurysm—this technique is broadly known as embolization. One example of such an approach is the Guglielmi Detachable Coil which involves intra-aneurysmal occlusion of the aneurysm via a system which utilizes a platinum coil attached to a stainless steel delivery wire and electrolytic detachment. Thus, once the platinum coil has been placed in the aneurysm, it is detached from the stainless steel delivery wire by electrolytic dissolution. Specifically, the patient's blood and the saline infusate act as the conductive solutions. The anode is the stainless steel delivery wire and the cathode is the ground needle which is placed in the patient's groin. Once current is transmitted through the stainless steel delivery wire, electrolytic dissolution will occur in the uninsulated section of the stainless steel detachment zone just proximal to the platinum coil (the platinum coil is of course unaffected by electrolysis). Other approaches involve the use of materials such as cellulose acetate polymer to fill the aneurysm sac. While these endovascular approaches are an advance in the art, they are disadvantageous. Specifically, the risks of these endovascular approaches include rupturing the aneurysm during the procedure or causing a stroke due to distal embolization of the device or clot from the aneurysm. Additionally, concern exists regarding the long term results of endovascular aneurysm obliteration using these techniques. Specifically, there is evidence of intra-aneurysmal rearrangement of the packing material and reappearance of the aneurysm on follow-up angiography.

One particular type of brain aneurysm which has proven to be very difficult to treat, particularly using the surgical clipping or endovascular embolization techniques discussed above occurs at the distal basilar artery. This type of aneurysm is a weak outpouching, usually located at the terminal bifurcation of the basilar artery. Successful treatment of this type of aneurysm is very difficult due, at least in part, to the imperative requirement that all the brainstem perforating vessels be spared during surgical clip placement.

Unfortunately, there are occasions when the size, shape and/or location of an aneurysm make both surgical clipping and endovascular embolization not possible for a particular patient. Generally, the prognosis for such patients is not good.

A significant advance in art of endovascular aneurysm occlusion is described in International Publication Number WO 99/40873, published Aug. 19, 1999 and International Publication Number WO 00/47134, published Aug. 12, 2000 [both naming Marotta, et al.]. The Marotta device is highly advantageous since it can be navigated to the site of “hard to reach” aneurysms where blockage of the aneurysmal opening may be achieved resulting in obliteration of the aneurysm.

Despite this significant advance in the art, there is still room for improvement. For example, the Marotta device comprises a so-called “leaf portion” for blockage of the aneurysmal opening. Once properly aligned, the leaf portion is advantageously useful to occlude the aneurysm. However, delivery can be difficult when using conventional balloon dilation catheters, since these catheters are typically used to deliver stents which do not require a specific orientation of the stent in relation to the target body passageway. Further difficulties can be encountered when attempting to deliver and properly orient the Marotta device to a bifurcated bodypassageway.

Accordingly, it would be desirable to have a catheter adapted to deliver an orient and an endovascular prosthesis in a body passageway.

### DISCLOSURE OF THE INVENTION

It is an object of the present invention to provide a novel expandable dilation catheter.

It is another object of the present invention to provide a novel balloon dilation catheter.

It is another object of the present invention to provide a novel catheterization kit.

It is another object of the present invention to provide a novel endovascular prosthesis mounted balloon dilation catheter.

Accordingly, in one of its aspects, the present invention provides an expandable dilation catheter comprising:

a first tubular member disposed in a proximal portion of the portion of the catheter and a second tubular member disposed in a distal portion of the catheter, the first tubular member and the second tubular member being in a spaced relationship with respect to one another;

an expandable member disposed distally of the second tubular member; and a first lumen and a second lumen disposed in each of the first tubular member and in the second tubular member, the first lumen in communication with an interior of the expandable member and the second lumen for receiving a first guidewire, the first tubular member and second tubular member being interconnected by a coupling member.

In another of its aspects, the present invention provides a balloon dilation catheter comprising:

a first tubular member disposed in a proximal portion of the portion of the catheter and a second tubular member disposed in a distal portion of the catheter, the first tubular member and the second tubular member being in a spaced relationship with respect to one another;

a balloon member disposed distally of the second tubular member; and

a first lumen, a second lumen and a third lumen disposed in each of the first tubular member and in the second tubular member, the first lumen in communication with an interior of the expandable member, the second lumen for receiving a first guidewire and the third lumen for receiving a second guidewire;

wherein the first tubular member and second tubular member are interconnected by at least one of the first lumen, the second lumen and the third lumen.

In another of its aspects, the present invention provides a catheterization kit comprising:

a guide catheter;

a pair of guidewires; and

a balloon dilation catheter comprising first tubular member disposed in a proximal portion of the portion of the catheter and a second tubular member disposed in a distal portion of the catheter, the first tubular member and the second tubular member being in a spaced relationship with respect to one another; a balloon member disposed distally of the second tubular member; and a first lumen, a second lumen and a third lumen disposed in each of the first tubular member and in the second tubular member, the first lumen in communication with an interior of the expandable member, the second lumen for receiving a first guidewire and the third lumen for receiving a second guidewire; wherein the first tubular member and second tubular member are interconnected by at least one of the first lumen, the second lumen and the third lumen.

In yet another of its aspects, the present invention provides an endovascular prosthesis-mounted balloon-catheter-comprising:

a first tubular member disposed in a proximal portion of the portion of the catheter and a second tubular member disposed in a distal portion of the catheter, the first tubular member and the second tubular member being in a spaced relationship with respect to one another;

a balloon member disposed distally of the second tubular member;

an expandable endovascular prosthesis mounted on the balloon member; and

a first lumen and a second lumen disposed in each of the first tubular member and in the second tubular member, the first lumen in communication with an interior of the expandable member and the second lumen for receiving a first guidewire, the first tubular member and second tubular member being interconnected by a coupling member.

Thus, the present inventors have discovered a catheter which may be used advantageously to deliver an endovascular prosthesis to a target body passageway and orient the prosthesis with respect to the body passageway. The present catheter is advantageous for delivery and orientation of an endovascular prosthesis such as the Marotta device referred to hereinabove. A feature of the present catheter is the presence of two tubular members which are spaced apart and interconnected by a coupling member. The nature of the coupling member is not particularly restricted provided that it allows relatively easier torquing or twisting of the spaced apart tubular members compared with a single, continuous tubular member. In one embodiment, this may be achieved by selecting the coupling member to have a cross-sectional diameter less than that of both of the tubular members. Preferably, the coupling member is in the form of one or more of the lumen used to inflate the expandable member (e.g., the balloon) on the distal end of the catheter and/or to receive the guidewire(s) used to navigate the catheter to the target body passageway.

### BRIEF DESCRIPTION OF THE DRAWINGS

Embodiments of the present invention will be described with reference to the accompanying drawings, in which:

FIG. 1 illustrates a perspective view of a preferred embodiment of the present catheter.

FIG. 2 illustrates an enlarged view of region A in FIG. 1.

FIGS. 3-7 illustrate sectional views along lines through VII-VII, respectively, in FIG. 2.

FIG. 8 illustrates an endovascular prosthesis which may delivered using the catheter illustrated in FIG. 1.

FIG. 9 illustrates mounting of the endovascular prosthesis of FIG. 8 on the catheter of FIG. 1.

FIG. 10 illustrates delivery of the endovascular prosthesis of FIG. 8 using the catheter of FIG. 1 to a bifurcated body passageway comprising an aneurysm.

FIG. 11 illustrates the bifurcated body passageway of FIG. 10, in perspective view, after deployment of the endovascular prosthesis of FIG. 8.

### BEST MODE FOR CARRYING OUT THE INVENTION

While various preferred embodiments of the present catheter will be described with reference to the Marotta endovascular prosthesis referred to hereinabove, this is for illustrative purposes only. Those of skill in the art will immediately recognize that the present catheter may be used to advantageously deliver and orient other endovascular prosthesis where it is desirable to orient the prosthesis in a particular manner.

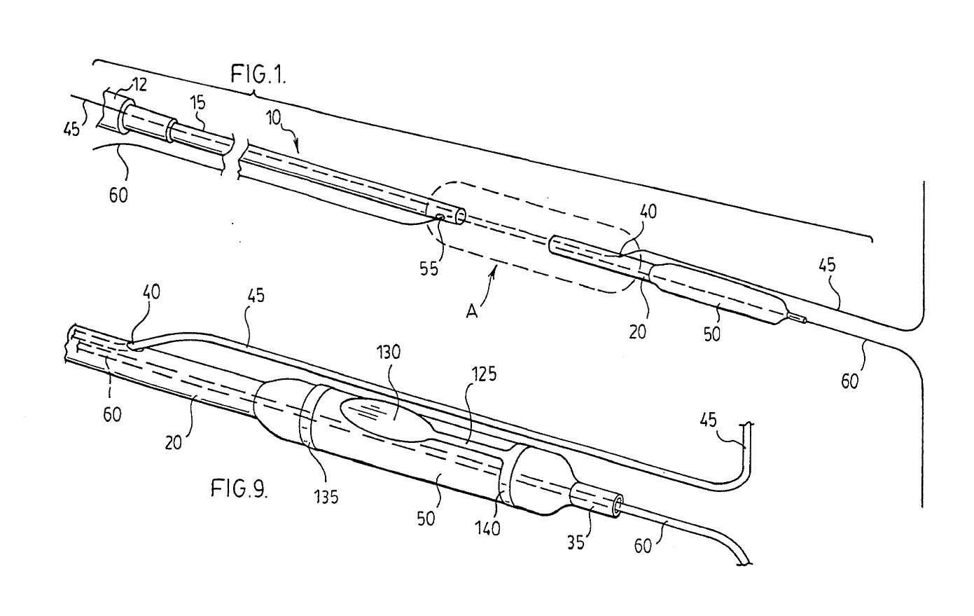

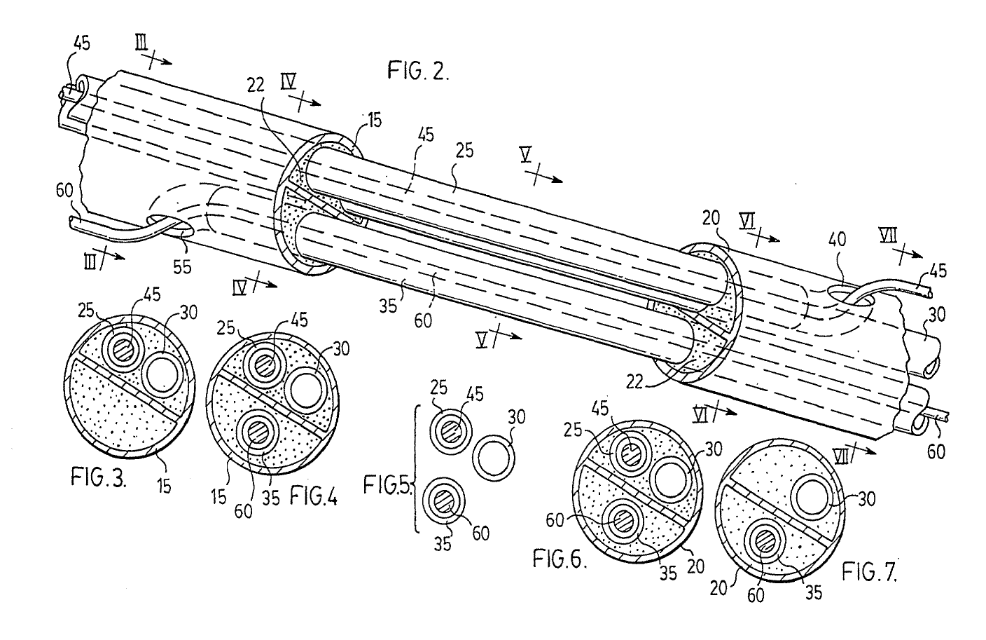

With reference to FIGS. 1-7, there is illustrated a balloon dilation catheter 10. Balloon catheter 10 comprises a first tubular member 15 and a second tubular member 20. Disposed at a proximal portion of first tubular member 15 is a Luer lock 12 (only a portion is illustrated) or similar device. First tubular member 15 and second tubular member 20 are of similar design, each comprising a so-called “double-D” cross-section with each “D” comprising a passageway—this can be seen particularly in FIGS. 2-4, 6 and 7.

First tubular member 15 and second tubular member 20 are interconnected by a trio of lumen 25,30,35. As illustrated, lumen 25,30,35 serve to space apart first tubular member 15 and second tubular member 20. Preferably, longitudinal spacing is less than about 10 cm, more preferably in the range of from about 1 cm to about 8 cm, most preferably in the range of from about 1 cm to about 5 cm. Lumen 25,30,35 are secured to first tubular member 15 and to second tubular member 20 by an adhesive 22.

Lumen 25 extends throughout first tubular member 15 into a portion of second tubular member 20. Thus, the proximal end of lumen 25 exits from Luer lock 12 in a conventional manner. Second tubular member 20 comprises an opening 40 in communication with lumen 25. As illustrated, lumen 25 receives a guidewire 45 which emanates from opening 40.

An expandable balloon 50 is secured to the distal end of second tubular member 20. The nature of balloon 50 and connection to second tubular member 20 is conventional and within the purview of a person skilled in the art.

Lumen 30 extends through first tubular member 15, second tubular member comprises a distal opening (not shown) in communication with an interior of balloon 50. The proximal end of lumen 30 exits from Luer lock 12 in a conventional manner. Thus, those of skill in the art will recognize the lumen 30 is a so-called inflation lumen used for inflation and deflation of balloon 50.

Lumen 35 extends from a portion of first tubular member 15 through second tubular member 20 and emanates from balloon 50. First tubular member 15 comprises an opening 55 in communication with lumen 35. As illustrated, lumen 35 receives a guidewire 60 through opening 55. Guidewire 60 is the portion of lumen 35 which emanates from balloon 50.

Lumen 25 contains guidewire 45 in a so-called “over-the-wire” configuration whereas lumen 35 contains guidewire 60 in a so-called “monorail” configuration. The use of the “monorail” configuration facilitates relatively rapid exchange of guidewire 60—see, for example, U.S. Pat. No. 4,748,982 [Horzewski, et al.] and the references cited therein for a general discussion on “monorail” delivery systems and rapid exchange of guidewires using such a system. It is, of course, possible to modify catheter 10 such that lumen 35 contains guidewire 60 in an “over-the-wire” configuration, in effecting yield a “double over-the-wire” configuration.

As will be appreciated by those of skill in art, first tubular member 15 and second tubular member 20 are disposed in a spaced relationship (i.e., similar to a single tubular member with a discontinuous portion) and are interconnected to each other by lumen 25,30,35. This allows for first tubular member 15 and second tubular member 20 to be torqued or twisted with respect to one another relatively easy compared to a construction where a single, continuous tubular member is used (i.e., no discontinuous portion). This added a relative degree of freedom between first tubular member 15, and second tubular member 20 facilitates orientation of an endovascular prosthesis mounted on balloon 50 as will be described in more detail hereinbelow.

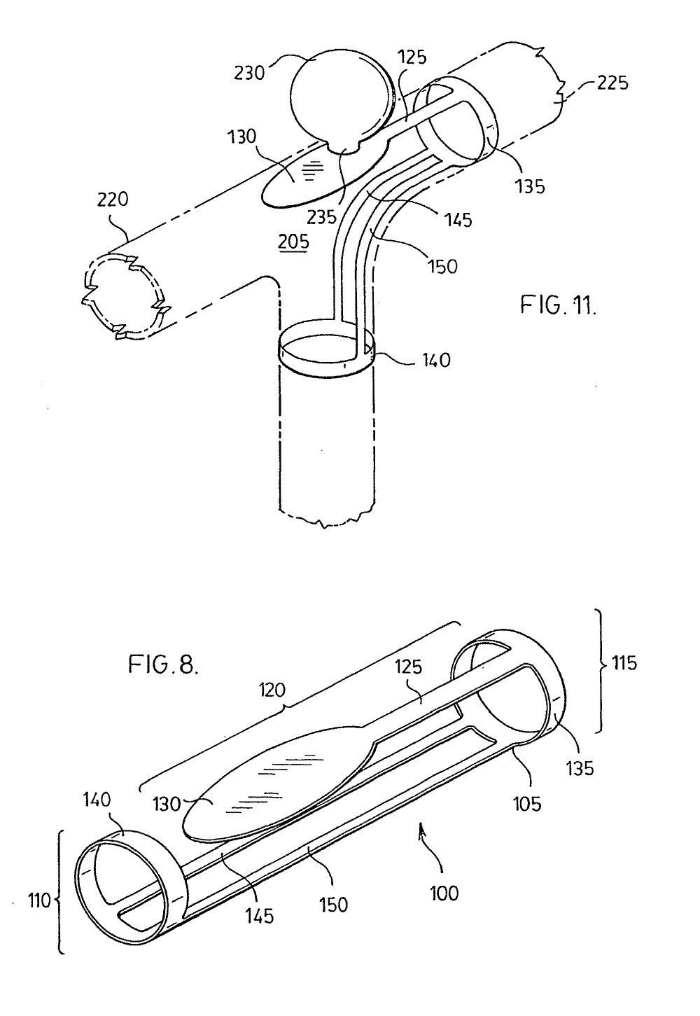

With reference to FIG. 8 there is of endovascular prosthesis 100 of similar construction as the Marotta device described hereinabove. Endovascular prosthesis 100 is constructed of a body 105. Body 105 comprises a proximal end 110 and a distal end 115. Endovascular prosthesis 100 further comprises a leaf portion 120 attached to body 105. As illustrated, leaf portion 120 comprises a neck 125 and a head 130. Head 130 is wider than neck 125. In the illustrated embodiment, head 130 of leaf portion 120 points away from distal end 115 (i.e., head 130 of leaf portion 120 points toward proximal end 110).

Body 105 further comprises a pair of rings 135,140 which are interconnected by a pair of struts 145,150. In the illustrated embodiment leaf portion 120 is connected to ring 135. Struts 145,150 preferably are dimensioned to confer to prosthesis 100 sufficient integrity while maximizing flexibility to provide enhanced navigation. The purpose of struts 145,150 is to interconnect rings 135,140 while allowing prosthesis 100 to be sufficiently flexible such that it can be navigated to the target body passageway yet be sufficiently expandable such that it can be fixed at the proper location in target body passageway. Struts 145,150 are not particularly important during expansion of prosthesis 100 (i.e., after the point in time at which prosthesis 100 is correctly positioned). Further, as will be apparent to those of skill in the art, leaf portion 120 is independently moveable with respect to proximal end 110 and distal end 115 of prosthesis 100 (in the illustrated embodiment, leaf portion 120 is independently moveable with respect to rings 135,140).

With reference to FIG. 9, prosthesis 100 is mounted on balloon 50 of catheter 10 in a conventional manner. For example, rings 135,140 may be crimped on balloon 50 of catheter 10. As shown, prosthesis 100 is mounted on balloon 50 such that neck 125 and 130 of leaf portion 120 are longitudinally aligned with opening 40 in second tubular member.

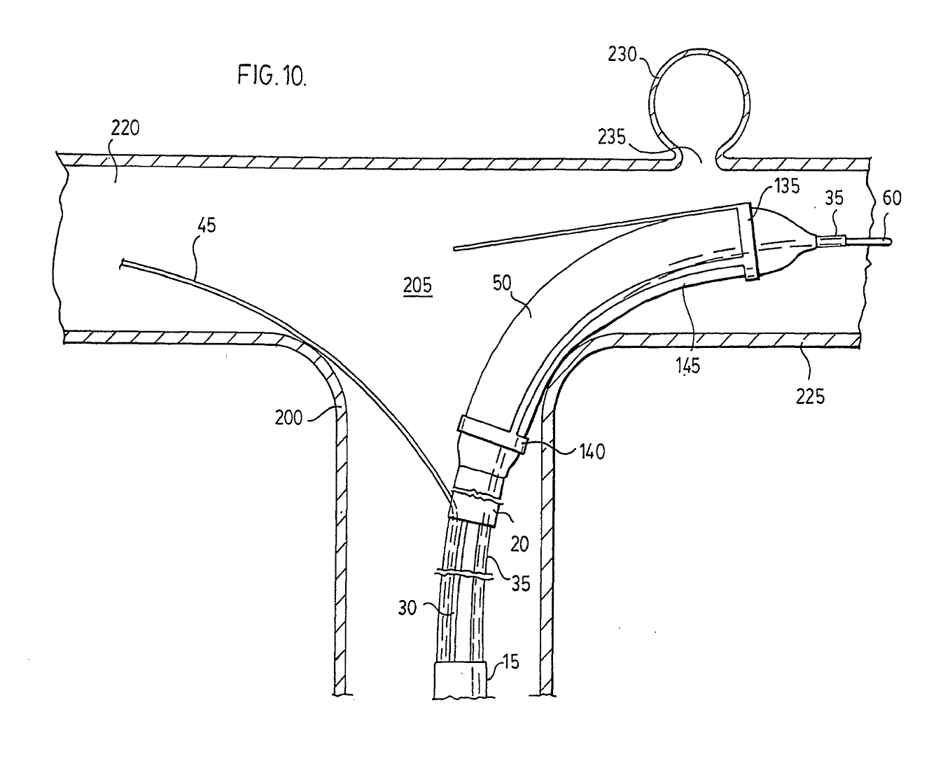

With reference to FIG. 10, delivery and deployment of prosthesis 100 mounted on balloon 50 of catheter 10 will be described.

Thus, there is illustrated a basilar artery 200 which terminates at a junction 205 which bifurcates into a pair of secondary arteries 220,225. Interposed between junction 205 and secondary artery 225 is an aneurysm 230. Aneurysm 230 has an opening 235 (shown enlarged for illustrative purposes only) through blood enters and sustains aneurysm 230. In the illustrated embodiment, opening 235 of aneurysm 230 is generally located on the superior surface of the arterial wall.

Guidewires 45,60 are delivered to secondary arteries 220,225, preferably using the guidewire delivery system described in International Publication Number WO 00/07525, published Feb. 17, 2000 [Ricci, et al.].

Next catheter 10 having prosthesis 100 mounted on balloon 50 (FIG. 9) is advanced over delivered guidewires 45,60 using the configuration illustrated in FIG. 1. As balloon 50 approaches junction 205 first tubular member sustains a natural torquing or twisting action as a result of alignment of guidewire 45 occurring with the approach opening 40. This torquing or twisting action is conveyed to lumen 25,30,35 and then to second tubular member 20. In response to the received torquing or twisting action, second tubular member naturally assumes a position in which lumen 25,30,35 are relatively untwisted and the portion of guidewire 45 emanating from opening 40 and the adjacent portion of catheter 10 are relatively untwisted. The combination of: (i) longitudinal alignment of opening 40 and leaf portion 120 of prosthesis 110, and (ii) spacing of apart of first tubular member 15 and second tubular member 20, advantageously facilitates the “untwisting” effect with the result that leaf portion 120 of prosthesis becomes oriented into substantial alignment with opening 235 of aneurysm 230.

Once endovascular prosthesis 100 is in the correct position, balloon 50 is expanded thereby exerting radially outward forces on rings 135,140. Initially, this results in expansion of ring 140 against the wall of both of basilar artery 200 and expansion of ring 135 in secondary artery 220. As expansion of balloon 50 continues, a portion of balloon 50 urges against neck 125 and head 130 of leaf portion 120 resulting in urging of leaf portion 120 against the walls of secondary artery 220 in a manner which results in blocking of opening 235 of aneurysm 230.

Next, balloon 50 is deflated and, together with guidewires 45,60, withdrawn from endovascular prosthesis 100. In the illustrated embodiment, endovascular prosthesis 100 is secured in position by rings 135,140 being urged against the walls of secondary artery 220 and basilar artery 200, respectively. Further, in the illustrated embodiment, leaf portion 120 is secured in position by a combination of forces against it by the flow of the blood into junction 205 and the inherent forces upon flexure of body 105 to navigate distal end 115 into secondary artery 220. Once leaf portion 120 blocks opening 35, aneurysm 30 is obliterated thereafter—see FIG. 11.

If opening 235 of aneurysm 230 is offset with respect to the superior surface of the arterial wall, the angle of such offset may be determined by a person skilled in cerebral angiography techniques, including 3-D rendering of the vascular anatomy in question. Once the anatomical angle of offset is determined, prosthesis 100 may be mounted on balloon 50 such that neck 125 and head 130 of leaf portion 120 are longitudinally offset from opening 40 in second tubular member 20 by a similar angle. This facilitates predictable aposition of leaf portion 120 over opening 235 of aneurysm 230.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. For example, will the tubular member illustrated with reference to the preferred embodiments comprises a so-called double-D cross-section, its possible to use tubular members with other cross-sections such as o-D (i.e., one passageway having a circular cross-section and the other having a D-shaped cross-section) and the like. It is possible to have the tubular member comprise individual lumen. Further, while the illustrated embodiments relate to a specific embodiment of the Marotta device referred to above, it is possible to advantageously use the present catheter with any endovascular prosthesis which should be specifically oriented with respect to the target body passageway. This includes stents and other expandable prosthesis disclosed in the Marotta, et al. International patent applications referred to above—e.g., it is possible to construct the prosthesis using a single expandable anchoring means (e.g., expandable tubular element, etc.) or 3 or more expandable anchoring means (e.g., expandable tubular elements, etc.). It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. A expandable dilation catheter comprising:
a first tubular member disposed in a proximal portion of the portion of the catheter and a second tubular member disposed in a distal portion of the catheter, the first tubular member and the second tubular member being in a longitudinally spaced relationship with respect to one another;
an expandable member disposed distally of the second tubular member; and
a first lumen and a second lumen disposed in each of the first tubular member and in the second tubular member, the first lumen in communication with an interior of the expandable member and the second lumen for receiving a first guidewire, the first tubular member and second tubular member being interconnected by a coupling member.

2. The expandable dilation catheter defined in claim 1, wherein the coupling member comprises the first lumen.

3. The expandable dilation catheter defined in claim 1, wherein the coupling member comprises the second lumen.

4. The expandable dilation catheter defined in claim 1, wherein the coupling member comprises each of the first lumen and the second lumen.

5. The expandable dilation catheter defined in claim 1, further comprising a third lumen disposed in each of the first tubular member and the second tubular member, the third lumen for receiving a second guidewire.

6. The expandable dilation catheter defined in claim 5, wherein the coupling member comprises the third lumen.

7. The expandable dilation catheter defined in claim 1, wherein the first lumen extends along substantially the entire length of the first tubular member.

8. The expandable dilation catheter defined in claim 1, wherein the first lumen extends along a portion of the length of the second tubular member.

9. The expandable dilation catheter defined in claim 1, wherein the second tubular member comprises a first aperture from which the first guidewire may exit the second lumen.

10. The expandable dilation catheter defined in claim 1, wherein the third lumen extends along substantially the entire length of first tubular member.

11. The expandable dilation catheter defined in claim 1, wherein the third lumen extends along a portion of the length of the first tubular member.

12. The expandable dilation catheter defined in claim 11, wherein the first tubular member comprises a second aperture into which the second guidewire may enter the third lumen.

13. The expandable dilation catheter defined in claim 12, wherein the first aperture and the second aperture are substantially opposed in a cross-section of the catheter.

14. The expandable dilation catheter defined in claim 1, wherein the third lumen extends along substantially the entire length of the second tubular member.

15. The expandable dilation catheter defined in claim 1, wherein the third lumen extends through a distal end of the expandable member.

16. The expandable dilation catheter defined in claim 1, wherein first tubular member comprises a first passageway and a second passageway.

17. The expandable dilation catheter defined in claim 1, wherein the second tubular member comprises a first passageway and a second passageway.

18. The expandable dilation catheter defined in claim 1, wherein each of the first tubular member and the second tubular member comprise a first passageway and a second passageway.

19. The expandable dilation catheter defined in claim 16, wherein the first lumen and the second lumen are disposed in the first passageway.

20. The expandable dilation catheter defined in claim 16, wherein the third lumen is disposed in the second passageway.

21. The expandable dilation catheter defined in claim 1, wherein the expandable member comprises a balloon portion.

22. The expandable dilation catheter defined in claim 1, wherein the first tubular member and the second tubular member have a substantially circular cross-section.

23. A balloon dilation catheter comprising:
a first tubular member disposed in a proximal portion of the portion of the catheter and a second tubular member disposed in a distal portion of the catheter, the first tubular member and the second tubular member being in a longitudinally spaced relationship with respect to one another;
a balloon member disposed distally of the second tubular member; and
a first lumen, a second lumen and a third lumen disposed in each of the first tubular member and in the second tubular member, the first lumen in communication with an interior of the expandable member, the second lumen for receiving a first guidewire and the third lumen for receiving a second guidewire;
wherein the first tubular member and second tubular member are interconnected by at least one of the first lumen, the second lumen and the third lumen.

24. The balloon dilation catheter defined in claim 23, wherein the first tubular member and the second tubular member are interconnected by at least two of the first lumen, the second lumen and the third lumen.

25. The balloon dilation catheter defined in claim 23, wherein the first tubular member and the second tubular member are interconnected by each of the first lumen, the second lumen and the third lumen.

26. The balloon dilation catheter defined in claim 23, wherein the first lumen extends along substantially the entire length of the first tubular member.

27. The balloon dilation catheter defined in claim 23, wherein the first lumen extends along a portion of the length of the second tubular member.

28. The balloon dilation catheter defined in claim 23, wherein the second tubular member comprises a first aperture from which the first guidewire may exit the second lumen.

29. The balloon dilation catheter defined in claim 23, wherein the third lumen extends along substantially the entire length of first tubular member.

30. The balloon dilation catheter defined in claim 23, wherein the third lumen extends along a portion of the length of the first tubular member.

31. The balloon dilation catheter defined in claim 30, wherein the first tubular member comprises a second aperture into which the second guidewire may enter the third lumen.

32. The balloon dilation catheter defined in claim 31, wherein the first aperture and the second aperture are substantially opposed in a cross-section of the catheter.

33. The balloon dilation catheter defined in claim 23, wherein the third lumen extends along substantially the entire length of the second tubular member.

34. The balloon dilation catheter defined in claim 23, wherein the third lumen extends through a distal end of the expandable member.

35. The balloon dilation catheter defined in claim 23, wherein first tubular member comprises a first passageway and a second passageway.

36. The balloon dilation catheter defined in claim 23, wherein the second tubular member comprises a first passageway and a second passageway.

37. The balloon dilation catheter defined in claim 23, wherein each of the first tubular member and the second tubular member comprise a first passageway and a second passageway.

38. The balloon dilation catheter defined in claim 23, wherein the first lumen and the second lumen are disposed in the first passageway.

39. The balloon dilation catheter defined in claim 23, wherein the third lumen is disposed in the second passageway.

40. The balloon dilation catheter defined in claim 23, wherein the first tubular member and the second tubular member have a substantially circular cross-section.

41. A catheterization kit comprising:
a guide catheter;
a pair of guidewires; and
the balloon dilation catheter defined in 23.

42. An endovascular prosthesis-mounted balloon catheter comprising:
a first tubular member disposed in a proximal portion of the portion of the catheter and a second tubular member disposed in a distal portion of the catheter, the first tubular member and the second tubular member being in a longitudinally spaced relationship with respect to one another;
a balloon member disposed distally of the second tubular member;
an expandable endovascular prosthesis mounted on the balloon member; and
a first lumen and a second lumen disposed in each of the first tubular member and in the second tubular member, the first lumen in communication with an interior of the expandable member and the second lumen for receiving a first guidewire, the first tubular member and second tubular member being interconnected by a coupling member.

43. The balloon catheter defined in claim 42, wherein the endovascular prosthesis comprises an expandable portion having attached thereto a leaf portion.

44. The balloon catheter defined in claim 43, wherein the leaf portion of the endovascular prosthesis is in substantial longitudinal alignment with the first aperture.
